(19) United States
(12) Patent Application Publication
(10) Pub. No.: US 2002/0032389 A1
(43) Pub. Date: Mar. 14, 2002

(54) Feminine self-sampling device and method

(76) Inventor: Arthur M. Fournier, Miami, FL (US)

Correspondence Address: MALLOY & MALLOY, P.A., 2800 S.W. Third Avenue, Historic Coral Way, Miami, FL, 33129, US

(21) Appl. No.: 09/867,337

(22) Filed: May 29, 2001

Related U.S. Application Data
(63) Continuation-in-part of application No. 09/716,648, filed on Nov. 20, 2000.
Application No. 09/716,648 is a continuation-in-part of application No. 09/206,760, filed on Dec. 7, 1998, now Pat. No. 6,155,990.

(51) Int. Cl. A61B 10/00

(52) U.S. Cl. 600/572; 600/562; 600/573; 604/330

(57) ABSTRACT

A female specimen gathering device is disclosed which can be self administered by women to collect specimens from the vaginal cavity. In particular, the device includes an insertion conduit structured to be at least partially introduced into the vaginal cavity of the female and a collection element housed within the insertion conduit to ultimately collect the specimen. A handle coupled with the collection element is provided to protrude the collection element from the insertion conduit and cause it to engage the sample collection location in order to actively collect a specimen therefrom. A fixative container is further provided and is structured to receive the collection element with the collected specimen, a multi-compatible fixative fluid being maintained in the fixative container in order to effectively preserve both fluid and solid types of specimen for a variety of different test procedures that permit testing for a variety of different conditions.

### CLAIM OF PRIORITY

The present application is a continuation-in-part application of previously filed, now pending application having Ser. No. 09/716,648 filed on Nov. 20, 2000, which is a continuation-in-part of issued U.S. Pat. No. 6,155,990, issued Dec. 5, 2000, the contents of both of which are incorporated herein by reference.

### BACKGROUND OF THE INVENTION

1. Field of the Invention

Screening for cervical cancer in women using cytological techniques, as well as the screening for various other vaginal related diseases, conditions and infections has been possible for many years, the present invention, however, relates to a self-sampling device which is easy and convenient to utilize by a women herself, without direct viewing requirements, such that the woman can effectively obtain a sample to be tested in privacy and with minimal discomfort. Moreover, the present device and method allow for a single sampling to be performed and contained in a manner that allows for a variety of different test procedures to be implemented in order to screen for the presence of many different conditions, including cytology, micro-array assays and tumor markers.

2. Description of the Related Art

In spite of the progress that has been made over the years in connection with the detection and testing for various illnesses, infections and other conditions associated with the vagina and/or cervix of a woman, there still remain a number of problems with the present technology. For example, conventional pap tests show a high percentage of smears of undetermined significance that requires further testing. This problem has led to the development of “thin prep” technology. Thin prep technology requires that cells be immersed in fixative and centrifuged prior to analysis. Other advances in diagnostic technology are the discovery of DNA probes for human papilloma virus (HPV), the causative agent of cervical cancer, and for chlamydia, a common infection in women. Test for HPV may soon replace conventional pap smears as the initial screening test for cervical cancer.

Unfortunately, given the present technology, collection of specimens, such as cytology specimens to screen for cancer, currently requires a speculum examination. Such an Examination, which is frequently uncomfortable and embarrassing for women, requires direct viewing into the vaginal cavity, which for purposes of this application is intended to include the vagina, cervix, and surrounding areas, and as such must be performed by a physician or nurse practitioner, thus also adding to the expense and inconvenience. In such circumstances the specimen obtained is applied directly to a glass slide, which is not compatible with automated cytologic analysis or necessary for HPV assay. The same problems of discomfort, embarrassment, expense and processing also apply to the obtaining of specimens to diagnose vaginal infections such as candidiasis, gonorrhea, human papilloma virus and chlamydia.

In an attempt to minimize the problems associated with traditional sampling techniques, a number of improved sampling devices were developed. Unfortunately, most such sampling devices were designed prior to the invention of thin prep and HPV assay technologies, and are generally of minimal if any effect for such testing, or were designed specifically to obtain a specimen in the setting of a conventional speculum examination, which does not alleviate some of the primary problems associated with speculum testing. Given these problems, there is still a need for an improved, inexpensive self sampling device which asymptomatic or symptomatic women can use in the privacy of their home at any time, that is adaptable to automated cytology methods (thin smear), HPV assay and microbial culture, and does not require direct viewing or third party assistance to obtain an effective and multi-purpose sample capable of aiding in the detection of a variety of different infections, illnesses, conditions, etc. This application discloses just such an improvement.

### SUMMARY OF THE INVENTION

Accordingly, the present invention is directed towards feminine self sampling device that will allow for the self sampling of cervical or vaginal specimens from the interior of the vaginal cavity, without direct viewing. The device will be inexpensive to produce, easy to destroy after use, and easy for women to use to perform a variety of testing, all with the single device and with a single specimen collection procedure. Furthermore, a preferred embodiment of the present device allows for the safe transportation of either solid of fluid specimens to laboratories for analysis, and is easily adaptable for centrifugation and thin-film preparation, or DNA probe for HPV, which is a technological improvement over the older direct preparation from a smear. Moreover, the present device may also be used to obtain specimens for culture or microbiologic assay, thus making it useful in the diagnosis of vaginal infections and for epidemiologic studies of sexually transmitted diseases. Indeed, the device and method offer advantages over brushes in the setting of direct visualization of the cervix, particularly when the patient is pregnant.

These and other features and advantages of the present invention will become more clear when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a perspective view of the self sampling device, taken from one side and below.

FIG. 2 is a cross-sectional view demonstrating the internal design.

FIG. 3 is a diagram demonstrating how the device is designed to fit and seal a standard cylindrical tube, in order to process the specimen using thin-film cytology.

FIG. 4 is an alternative embodiment including a collapsible handle.

FIG. 5 is an alternative embodiment of the present invention including a removable collection element.

Like reference numerals refer to like parts throughout the several views of the drawings.

### DETAILED DESCRIPTION OF THE PREFERRED EMBODIMENT

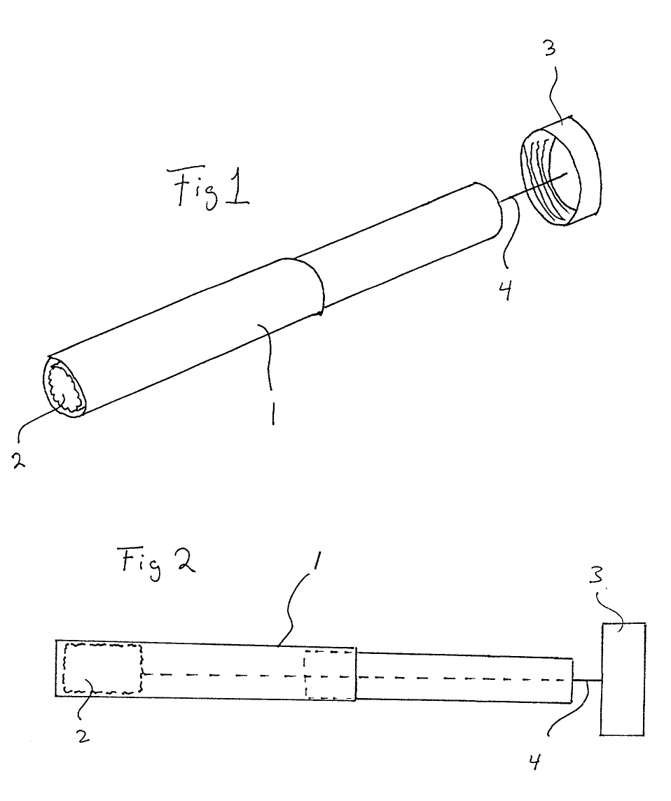

Please refer to FIGS. 1-5 for a complete understanding of this invention. The drawings represented the current best conceptualization of the design, and is not to be construed as a restricting or limitation of its scope.

As can be seen in the embodiment of FIGS. 1 and 2, the device may be considered an assembled unit consisting of subunits such as: a) an insertion conduit, such as the illustrated “telescoping” cylindrical tube (1) which houses, b) the specimen-gathering collection element, such as a sponge (2). The sponge is preferably secured to an elongate handle (4), that is capable of extending the collection element (2) into the vaginal cavity along a path defined and guided by the insertion conduit (1). Also as shown in the embodiment of the figures, a cap (3) is provided and may define an exterior portion of the handle (4) may also be provided, the cap further functioning to shield and later contain the collection element (2) as will be described.

Looking in further detail at the insertion conduit (1), it may be configured as a cylindrical sheath constructed of medical-quality cardboard and coated with wax or Teflon to allow for minimal friction on insertion. Furthermore, although it is contemplated that a single tube segment of sufficient length may be provided, in the illustrated embodiment, so as to achieve a more compact configuration, the telescoping type tube configuration is preferably employed. In either embodiment, the insertion conduit functions to shield and protect the collection element during insertion. For example, although it is contemplated that the insertion conduit (1) is first introduced into the vaginal cavity, and subsequently, the collection element (2) is passed therethrough, in the illustrated embodiment, the collection element (2) is contained by the insertion conduit (1) as the complete unit is inserted and extended into the vaginal cavity. For example, with the “telescope” fully extended and the collection element (2) may be housed in the anterior chamber of the insertion conduit (1) during insertion, thus acting as a sheath to protect the collection element.

Looking further at the insertion conduit (1), it is preferably sized and configured so as to define a path for the collection element (2), and thereby guide the collection element (2) to a desired sample collection location. By effectively sizing the insertion conduit to correspond the average insertion depth to reach the cervix (typically about 13 cm), the insertion conduit (1) maybe inserted into the vaginal cavity until resistance is met or until a predefined marking on the conduit itself. As a result, a user does not have to directly visualize the interior of the vaginal cavity, and the collection element (2) is guided directly to the sample collection location without undue manipulation that may lead to injury or discomfort.

With the insertion conduit (1) in place and effectively positioned to the guide the collection element (2), the collection element (2) may then be extended therefrom by the handle (4). For example, by grasping the posterior end of the insertion conduit (1) between two fingers and pressing on the protruding portion of the handle (3), the collection element (2) will extend into the vaginal vault, contacting the sample collection location.

Looking further to the handle (4) and as illustrated in FIG. 4, the handle itself may also be collapsible, either into its own sheath, as in the illustrated embodiment, and/or into the outer portion of the insertion conduit (1). Such an embodiment may be especially beneficial for effective packaging and/or for the introduction of both the handle (4) and the collection element (2) into a fixative container of a smaller size.

The collection element (2) may be composed of several possible flexible, perhaps resilient materials, including, but not limited to cellulose, natural or artificial sponge material. As such, the collection element (2) preferably includes an at least, mildly abrasive quality that serves to exfoliate the collection location and dislodge cells to be gathered as part of the collected sample. Furthermore, the collection element (2) may have good adherent and mildly abrasive qualities, and in such an embodiment, the vacuum created by the sponge upon insertion should draw cells down from the cervical canal without the trauma that might be created by a traditional spatula. It also obviates the need for direct visualization of the cervical os. Of course, it is understood that the composition of the collection element may be modified under special circumstances; i.e., a more abrasive sponge may be warranted in screening for HIV inter-menses, and a more absorptive sponge is indicated during menses or to obtain specimens during abnormal uterine bleeding.

Looking in further detail to the collection of the specimen, after insertion of the insertion conduit (1) and extension of the collection element (2), the handle (4) may be rotated to exfoliate the surface and obtain the specimen. The collection element (2) may then be retracted in order to return it to the anterior chamber of the insertion conduit (1) prior to removal. In this manner the collection element (2) is shielded and the specimen preserved on the collection element as it is removed from the vaginal cavity. In the case of a telescoping insertion conduit, a rim on the inner tube and a groove on the inner surface of the outer tube may be provided to assure that the telescope mechanism does not collapse prior to the collection element returning to the anterior chamber.

In this preferred, illustrated embodiment, the overall length of the device may be approximately 15 cm, the insertion conduit (1) being about 13 cm when fully extended and disposed in an extended orientation. As such, the handle (4) which may be in the range of 8-18 cm long, and as indicated may be of a fixed dimension or extendable, is preferably about 14 cm when extended so as to provide for sufficient protrusion of the collection element from the insertion conduit for engagement with the patient. Moreover, in the illustrated embodiment, the width or diameter will be about 1.5 cm. These dimensions will accommodate the standard vaginal cavity depth of 12.7 cm for a mature woman, and are sufficiently narrow to allow for comfortable insertion. In the telescoping embodiment, the device will collapse to about 7 cm, so as to define a compressed orientation, allowing it to be easily inserted into a specimen tube (5) of about 11.5 cm in depth. As will be described, as an alternative, the collection element may be expulsed by itself into the fixative.

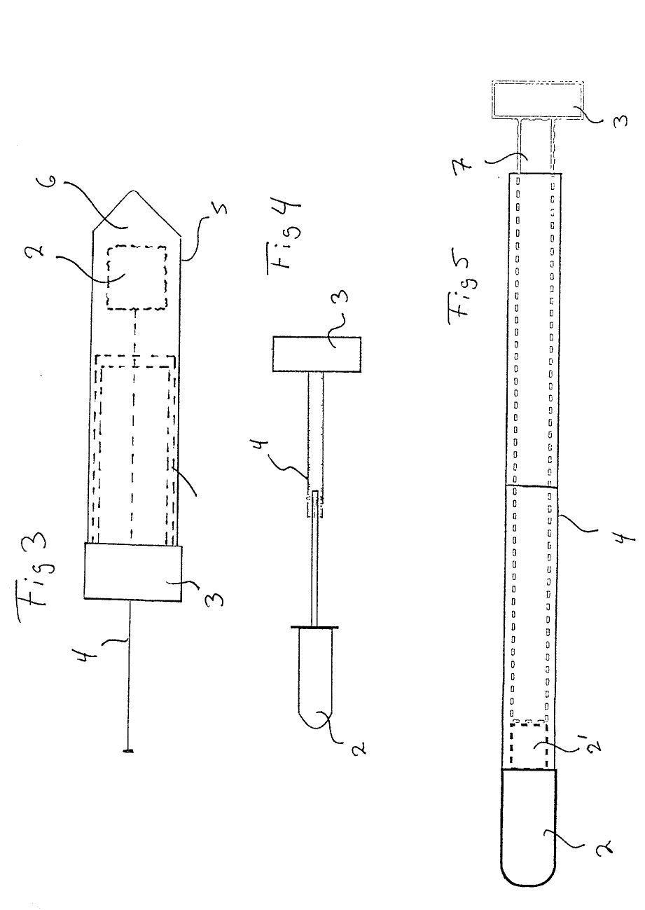

In particular, the present invention also preferably includes a fixative container (5) into which the collection element (2), with the sample contained thereon, is introduced. As will be described in further detail, the fixative container (5) includes a fixative (6) therein to preserve the specimen and into which the collection element may be immersed. In the illustrated embodiment, the entire collection portion of the device may be introduced into the fixative container (5), the collapsed insertion conduit (1) and/or handle (4) allowing for complete introduction thereof with the collection element exposed. Moreover, in the embodiment wherein the exterior portion (3) of the handle (4) is in the form of a screw on cap, the cap may be configured to form a tight seal on the fixative container (5). Also in such an embodiment, if the handle (4) is of a fixed size or a large collapsed size, it may extend out through the cap (see FIG. 3), its length of protrusion determined by the collapse of the telescope and the need for the collection element sponge to be freely immersed in the fixative (6). Of course, as an alternative embodiment, and as illustrated in FIG. 5, the collection element may be configured to be removable from the handle (4),and as such, once removed from the patient, the collection element may be placed inside the fixative container (5) separate from the handle (4) and remaining structure. In such an embodiment, a plunger element (7) may be provided and configured to be pushed by the user after collection of the sample so as to detatch the collection element (2) from the handle (4). As shown in FIG. 5, in such an embodiment the collection element may have an attachment segment (2′) that extends into the handle (4) so as to maintain secure engagement until removed, either directly by the user or by pushing of the plunger element (7). The collection element (2) may then be sealed in the fixative container by a conventional cap and a remainder of the structure discarded or sterilized for re-use.

As mentioned, the collection element (2) is preferably immediately immersed in the fixative after removal of the device from the patient, avoiding problems associated with drying. This fixative thereby preserves and maintains the collected specimen, allowing it to be maintained and/or transported for testing of the specimen. In this regard, home testing and/or transport to a laboratory for testing may be achieved. In either case, when testing is desired, the fixative container (5) is preferably agitated to liberate cells and/or fluid from the sponge, centrifuged and the supernatant discarded. The cellular contents may be removed and prepared as a thin-prep slide for either manual or automated cytologic examination using conventional cytologic techniques. As to fluid specimens, a culture test may be performed, and/or a molecular pathology assay type test may be performed. These include “wet mount”, KOH prep for fungal examinations or PCR assays for chlamydia and human papilloma virus, or other infectious agents such as human immunodeficiency and hepatitis viruses and tumor markers from a variety of cancers. The device can then be disposed of using “universal precautions” and incinerated. It should be noted that both the insertion conduit and the collection element may be used to obtain vaginal as well as cervical samples form what is generally defined as the vaginal cavity.

Looking in further detail to the fixative fluid (6) it is preferably configured to be both fluid and cell compatible so as to facilitate cytology and culture/micro-array assay testing from the same collected specimen, or from specimens collected utilizing the same techniques. As such, the fixative fluid may be said to be RNA friendly, and may comprise a fluid varying from 5% alcohol, to the commercially available saccomanno fluid that is composed of ethyl alcohol, isopropyl alcohol, methyl alcohol, polyethylene alcohol and MIBK.

Accordingly, although designed for self-sampling of cytologic specimens, it may have wide applications in the diagnosis of vaginal infections, in conducting epidemiologic studies of sexually transmitted diseases or in comparing the utility of PCR assay for human papilloma virus to conventional cytology as a screening test for cervical cancer. Indeed, the device may also be used for cell sampling of colon, oral, uterine and dermal tissues with only minor modifications, and if desired, processed in such a way as to allow for assay of cytology, infectious agents and tumor markers associated with oral, rectal and uterine cancer, among other possible conditions. As yet another alternative, the present device and method may also be utilized to brush or spatula to obtain specimens under direct vision. This application has special merit in the case of pregnant women, in whom brushing is contradicted. Finally, the insertion conduit (1) may be used for culture, micro-array assay or other tests to diagnose vaginal infections. Further, the present invention may also be utilized as an applicator of topical treatments and medications. For example, direct topical application of medication directly on the cervix has generally been found to be difficult due to the lack of an appropriate instrument that not only effectively reaches the application site, but also maintains the medication to be applied in a generally shielded state during inter-labial introduction. As such, it is recognized that the medication could be effectively employed by applying the medication to the collection element (2), inserting the shielded collection element into the vaginal cavity via the protective insertion conduit (1), and extending the collection element (2) into contact with the cervix or other treatment region so as to directly apply the medication.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A feminine self-sampling device comprising:
an insertion conduit, said insertion conduit structured to define an access path into a vaginal cavity of a female;
a collection element at least temporarily housed within said tube,
a handle coupled with said collection element and structured to protrude said collection element from said insertion conduit;
said insertion conduit further structured to guide said collection element towards a sample collection location within the vaginal cavity of the female so as to eliminate a direct visualization requirement; and
said collection element structured to engage the sample collection location and to collect a specimen therefrom.

2. The feminine self-sampling device recited in claim 1 wherein said handle is further structured to retract said collection element into said insertion conduit for protected removal from the vaginal cavity with a collected specimen.

3. The feminine self-sampling device recited in claim 1 wherein said collection element includes an abrasive surface structured to engage the sample collection location and remove and collect cells as said collected specimen from said sample collection location.

4. The feminine self-sampling device recited in claim 3 wherein said collection element is further structured to collect fluid from said sample collection location.

5. The feminine self-sampling device recited in claim 4 further comprising a fixative container into which said collection element containing said collected specimen is introduced subsequent to removal from the vaginal cavity, said fixative container including a fixative fluid contained therein and into which said collection element is immersed.

6. The feminine self-sampling device recited in claim 5 wherein said fixative fluid is both fluid and cell compatible so as to facilitate cytology and micro-array assay testing.

7. The feminine self-sampling device recited in claim 6 wherein said fixative fluid is RNA friendly.

8. The feminine self-sampling device recited in claim 6 wherein said fixative fluid is in liquid form and said collected specimen is structured to be retrieved from said collection element into said fixative fluid as a result of agitation of said collection element within said fixative fluid.

9. The feminine self-sampling device recited in claim 5 wherein said handle includes a cap, said cap structured to cooperatively engage said fixative container and enclose said collection element therein.

10. The feminine self-sampling device recited in claim 5 wherein said collection element is removably secured to said handle and is structured to be independently deposited in said fixative container.

11. The feminine self-sampling device recited in claim 10 wherein said handle includes a plunger assembly structured to separate said collection element from said handle.

12. The feminine self-sampling device recited in claim 5 wherein said fixative fluid includes approximately 5% alcohol.

13. The feminine self-sampling device recited in claim 5 wherein said fixative fluid includes a saccomanno fluid.

14. The feminine self-sampling device recited in claim 1 wherein said handle is structured to extend said collection element approximately 8-18 cm into said vaginal cavity so as to engage a cervix of the female as said sample collection location.

15. The feminine self-sampling device recited in claim 1 wherein said insertion conduit includes a telescoping tube structured to extend into said vaginal cavity.

16. The feminine self-sampling device recited in claim 1 wherein said handle includes a telescoping configuration.

17. A feminine self-sampling device comprising:
an insertion conduit, said insertion conduit structured to be at least partially introduced into a vaginal cavity of a female;
a collection element at least temporarily housed within said insertion conduit;
a handle coupled with said collection element and structured to protrude said collection element from said insertion conduit;
said collection element structured to engage the sample collection location and to actively collect a specimen therefrom;
a fixative container structured to receive said collection element with said collected specimen therein; and
a multi-compatible fixative disposed in said fixative container, said multi-compatible fixative structured to preserve both fluid and solid types of said collected specimen.

18. The feminine self-sampling device recited in claim 17 wherein said fixative is structured to facilitate cytologic testing on cells as said collected specimen.

19. The feminine self-sampling device recited in claim 17 wherein said fixative is structured to facilitate culture experimentation on said collected specimen.

20. The feminine self-sampling device recited in claim 17 wherein said fixative is structured to facilitate molecular pathology assay type testing.

21. The feminine self-sampling device recited in claim 17 wherein said collection element is at least partially abrasive so as to remove and collect cells from the sample collection location.

22. The feminine self-sampling device recited in claim 17 wherein said collection element is at least partially absorbent so as to collect fluids from the sample collection location.

23. A feminine self diagnosis and treatment device comprising:
an insertion conduit, said insertion conduit structured to be at least partially introduced into a vaginal cavity of a female;
a collection element structured to removably hold and contain a material thereon;
said insertion conduit defining a sheath structured to at least temporarily house and shield said collection element during at least one direction of inter-labial passage of said collection element; and
a handle coupled with said collection element and structured to protrude said collection element from said insertion conduit such that said collection element engages a cervix.

24. A method of obtaining a specimen from a vaginal cavity of a female comprising:
the female introducing a insertion conduit at least partially into the vaginal cavity;
the female extending a collection element from the introduced insertion conduit into the vaginal cavity without direct viewing and along a path guided by said insertion conduit;
the female engaging a cervix within the vaginal cavity with the collection element and affirmatively gathering a specimen in said collection element;
the female removing said collection element with said collected specimen from said vaginal cavity;
the female introducing said collection element with said collected specimen into a fixative and containing at least said collected specimen in said fixative; and
producing said contained collected specimen in said fixative for testing.

25. The method of obtaining a specimen recited in claim 24 wherein the female engaging the cervix within the vaginal cavity with the collection element and affirmatively gathering the specimen in said collection element further comprises absorbing a fluid specimen in said collection element.

26. The method of obtaining a specimen recited in claim 24 wherein the female introducing said collection element with said collected specimen into said fixative and containing at least said collected specimen in said fixative further comprises introducing a solid and a fluid collected specimen into a single, multi-compatible fixative.
